(19) United States
(12) Patent Application Publication
(10) Pub. No.: US 2024/0358615 A1
(43) Pub. Date: Oct. 31, 2024

(54) COMPOSITION FOR CARING FOR KERATIN MATERIALS

(71) Applicant: L'OREAL, Paris (FR)

(72) Inventors: Yu HUANG, Shanghai (CN); Lingling SUN, Shanghai (CN); Julien LABOUREAU, Shanghai (CN)

(73) Assignee: L'OREAL, Paris (FR)

(21) Appl. No.: 18/684,938

(22) PCT Filed: Sep. 23, 2021
(86) PCT No.: PCT/CN2021/119838

§ 371 Date: Feb. 20, 2024

(51) Int. Cl. A61K 8/60; A61K 8/37; A61Q 19/08

(52) U.S. Cl. CPC A61K 8/604; A61K 8/375; A61Q 19/08; A61K 2800/596

(57) ABSTRACT

The present invention provides a composition for a non-therapeutic method for caring for keratin materials. The composition contains in an aqueous phase: (i) at least one cosmetic active compound selected from C-glycosides of formula (I):

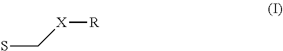

wherein R represents a saturated C₁ to C₁₀, alkyl radical which can optionally be substituted by at least one radical chosen from OH, COOH or COOR″₂, with R″₂ being a saturated C₁-C₄ alkyl radical, S represents a monosaccharide or a polysaccharide comprising up to 20 sugar units in pyranose and/or furanose form and of the L and/or D series, and X represents a radical chosen from the —CO—, —CH(OH)—, —CH(NH₂)—, —CH(NHCH₂CH₂CH₂OH)—, —CH(NHPh)- and —CH(CH₃)— groups, the S—CH₂—X bond represents a bond of an α or β C-anomeric nature; (ii) at least one surfactant selected from fatty acid esters of polyglycerol; and (iii) at least one surfactant selected from oxyalkylenated fatty acid esters of glycerol.

### TECHNICAL FIELD

The present invention relates to a cosmetic composition. In particular, the present invention relates to a composition for caring for keratin materials. The present invention also relates to a non-therapeutic method for caring for keratin materials.

### BACKGROUND ART

The skin is the protective barrier for the human body. It protects the interior of the body from physical injury (such as trauma) and biological injury (such as bacteria, viruses or fungi). The epidermis is a keratinized stratified pavimentous epithelium. Its mean thickness ranges from 60 to 100 μm and may reach 600 to 700 μm on the sole of the feet and the palm of the hands. It consists mainly of keratinocytes, but also other cells, and rests on a basal membrane that separates it from the dermis.

During the menopause, the skin undergoes changes in all its compartments, i.e. dermal and epidermal.

The main changes concern the dermis and are a decrease in the collagen content and in the thickness of the dermis. In menopausal women, this results in thinning of the skin and/or of the mucous membranes. Women then experience a sensation of “dry skin” or of taut skin and an accentuation of the surface wrinkles and fine lines is observed. The skin has a rough feel. Finally, the skin shows decreased suppleness.

The main changes concerning the epidermis are a decrease in keratinocyte differentiation, resulting in a deficit in the proteins matrix of the cornified cell, an increase in metalloproteinases, which are proteases that degrade the extracellular matrix and that participate in ageing of the skin, and also a decrease in the synthesis of various glycosaminoglycans.

The development of formulations dedicated to caring for and/or making up the skin and/or lips, is permanent.

A wide variety of cosmetic compositions have been used to care for the skin, for example, to combate the ageing of the skin. There are commercial products for antiaging of the skin comprising hydroxypropyl tetrahydropyrantriol. However, for some of them, the penetration of hydroxypropyl tetrahydropyrantriol into the skin is not good enough.

Thus, there is still a need to formulate a composition for caring for the skin, which can deliver hydroxypropyl tetrahydropyrantriol or the like effectively.

### SUMMARY OF THE INVENTION

The inventors have now discovered that it is possible to formulate compositions for caring for the skin, which can deliver hydroxypropyl tetrahydropyrantriol or the like effectively.

Accordingly, in a first aspect, the present invention provides a composition for caring for keratin materials comprising in an aqueous phase:
(i) at least one cosmetic active compound selected from C-glycosides of formula

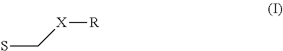

in which:
R represents a saturated C₁ to C₁₀, in particular C₁ to C₄, alkyl radical which can optionally be substituted by at least one radical chosen from OH, COOH or COOR″₂, with R″₂ being a saturated C₁-C₄ alkyl radical,
S represents a monosaccharide or a polysaccharide comprising up to 20 sugar units, in particular up to 6 sugar units, in pyranose and/or furanose form and of the L and/or D series, it being possible for the said monosaccharide or polysaccharide to be substituted by a hydroxyl group which is necessarily free and optionally one or more optionally protected amine functional group(s), and
X represents a radical chosen from the —CO—, —CH(OH)—, —CH(NH₂)—, —CH(NHCH₂CH₂CH₂OH)—, —CH(NHPh)- and —CH(CH₃)— groups and in particular a —CO—, —CH(OH)— or —CH(NH₂)— radical and more particularly a —CH(OH)— radical, the S—CH₂—X bond represents a bond of C-anomeric nature, which can be α or β, and also their physiologically acceptable salts, their solvates, such as the hydrates,
and their optical and geometrical isomers;
(ii) at least one surfactant selected from fatty acid esters of polyglycerol; and
(iii) at least one surfactant selected from oxyalkylenated fatty acid esters of glycerol.

In a second aspect, the present invention provides a non-therapeutic method for caring for keratin materials, comprising applying the composition according to the first aspect of the present invention to the keratin materials.

It was found that the composition of the present invention can deliver the cosmetic active compound to the keratin materials effectively.

It was also found that the composition of the present invention also can deliver a good skin sensory, for example has a stickiness acceptable for skincare products.

Other advantages of the present invention will emerge more clearly on reading the description and the examples that follow.

### BRIEF DESCRIPTION OF THE DRAWINGS

Implementations of the present invention will now be described, by way of example only, with reference to the attached figures, wherein:

FIG. 1 shows the Raman spectra of 400-2000 cm⁻¹ for hydroxypropyl tetrahydropyrantriol;

FIG. 2 shows the penetration profile of hydroxypropyl tetrahydropyrantriol for the composition of invention formula 1;

FIG. 3 shows the penetration profile of hydroxypropyl tetrahydropyrantriol for the composition of comparative formula 1; and

FIG. 4 shows the penetration profile of hydroxypropyl tetrahydropyrantriol for the composition of comparative formula 4.

### DETAILED DESCRIPTION OF THE INVENTION

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “between . . . and . . . ” and “ranging from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values which are capable of being changed according to the desired purpose as required.

For the purposes of the present invention, the term “keratin materials” is intended to cover human skin, mucous membranes such as the lips. Facial skin is most particularly considered according to the present invention.

All percentages in the present invention refer to weight percentage, unless otherwise specified.

### C-Glycosides of Formula (I)

According to the first aspect, the composition of the present invention comprises at least one cosmetic active compound selected from C-glycosides of formula (1):

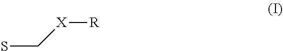

in which:
R represents a saturated C₁ to C₁₀, in particular C₁ to C₄, alkyl radical which can optionally be substituted by at least one radical chosen from OH, COOH or COOR″₂, with R″₂ being a saturated C₁-C₄ alkyl radical,
S represents a monosaccharide or a polysaccharide comprising up to 20 sugar units, in particular up to 6 sugar units, in pyranose and/or furanose form and of the L and/or D series, it being possible for the said monosaccharide or polysaccharide to be substituted by a hydroxyl group which is necessarily free and optionally one or more optionally protected amine functional group(s), and
X represents a radical chosen from the —CO—, —CH(OH)—, —CH(NH₂)—, —CH(NHCH₂CH₂CH₂OH)—, —CH(NHPh)- and —CH(CH₃)— groups and in particular a —CO—, —CH(OH)— or —CH(NH₂)— radical and more particularly a —CH(OH)— radical,
the S—CH₂—X bond represents a bond of C-anomeric nature, which can be α or β, and also their physiologically acceptable salts, their solvates, such as the hydrates, and their optical and geometrical isomers.

The C-glycosides of formula (1) of use for the implementation of the invention are in particular those for which R denotes a saturated linear C₁ to C₆, in particular C₁ to C₄, preferentially C₁ to C₂, alkyl radical and more preferably a methyl radical.

Mention may in particular be made, among the alkyl groups suitable for the implementation of the invention, of methyl, ethyl, isopropyl, n-propyl, n-butyl, t-butyl, isobutyl, sec-butyl, pentyl, n-hexyl, cyclopropyl, cyclopentyl or cyclohexyl groups.

According to one embodiment of the invention, use may be made of a C-glycoside compound corresponding to the formula (1) for which S can represent a monosaccharide or a polysaccharide comprising up to 6 sugar units, in pyranose and/or furanose form and of L and/or D series, the said mono- or polysaccharide exhibiting at least one necessarily free hydroxyl functional group and/or optionally one or more necessarily protected amine functional groups, X and R otherwise retaining all of the definitions given above.

Advantageously, a monosaccharide of the invention can be chosen from D-glucose, D-galactose, D-mannose, D-xylose, D-lyxose or L-fucose,
L-arabinose, L-rhamnose, D-glucuronic acid, D-galacturonic acid,
D-iduronic acid, N-acetyl-D-glucosamine or N-acetyl-D-galactosamine and advantageously denotes D-glucose, D-xylose, N-acetyl-D-glucosamine or L-fucose and in particular D-xylose.

More particularly, a polysaccharide of the invention comprising up to 6 sugar units can be chosen from D-maltose, D-lactose, D-cellobiose,
D-maltotriose, a disaccharide combining a uronic acid chosen from
D-iduronic acid or D-glucuronic acid with a hexosamine chosen from
D-galactosamine, D-glucosamine, N-acetyl-D-galactosamine or N-acetyl-D-glucosamine, an oligosaccharide comprising at least one xylose which can advantageously be chosen from xylobiose, methyl-β-xylobioside, xylotriose, xylotetraose, xylopentaose and xylohexaose and in particular xylobiose, which is composed of two xylose molecules linked via a 1-4 bond.

More particularly, S can represent a monosaccharide chosen from D-glucose, D-xylose, L-fucose, D-galactose or D-maltose and in particular D-xylose.

Preferably, use is made of a C-glycoside derivative of formula (1) for which:
R denotes an unsubstituted linear C₁-C₄, in particular C₁-C₂, alkyl radical, especially a methyl radical;
S represents a monosaccharide as described above and chosen in particular from D-glucose, D-xylose, N-acetyl-D-glucosamine or
L-fucose, and in particular D-xylose;
X represents a group chosen from —CO—, —CH(OH)— or —CH(NH₂)— and preferably a —CH(OH)— group.

The acceptable salts of the compounds described in the present invention comprise conventional non-toxic salts of the said compounds, such as those formed from organic or inorganic acids. Mention may be made, by way of example, of the salts of inorganic acids, such as sulfuric acid, hydrochloric acid. Mention may also be made of the salts of organic acids, which can comprise one or more carboxylic, sulfonic or phosphonic acid groups. Mention may in particular be made of propionic acid, acetic acid, terephthalic acid, citric acid and tartaric acid.

When the compound of formula (I) comprises an acid group, neutralization of the acid group(s) can be carried out with an inorganic base, such as LiOH, NaOH, KOH, Ca(OH)₂, NH₄OH, Mg(OH)₂ or Zn(OH)₂, or with an organic base, such as a primary, secondary or tertiary alkylamine, for example triethylamine or butylamine. This primary, secondary or tertiary alkylamine can comprise one or more nitrogen and/or oxygen atoms and can thus comprise, for example, one or more alcohol functional groups; mention may in particular be made of 2-amino-2-methylpropanol, triethanolamine, 2-(dimethylamino)propanol or 2-amino-2-(hydroxymethyl)-1,3-propanediol. Mention may also be made of lysine or 3-(dimethylamino)propylamine.

The solvates which are acceptable for the compounds described in the present invention comprise conventional solvates, such as those formed during the final stage of preparation of the said compounds due to the presence of solvents. Mention may be made, by way of example, of the solvates due to the presence of water or of linear or branched alcohols, such as ethanol or isopropanol.

Of course, according to the invention, a C-glycoside derivative corresponding to the formula (1) can be used alone or as a mixture with other C-glycoside derivatives and in any proportion.

A C-glycoside derivative which is suitable for the invention can in particular be obtained by the synthetic method described in the document WO 02/051828.

Mention may in particular be made, by way of non-limiting illustration of the C-glycoside compounds which are particularly suitable for the invention, of the following compounds:
C-β-D-xylopyranoside-n-propane-2-one,
C-αD-xylopyranoside-n-propan-2-one,
C-β-D-xylopyranoside-2-hydroxypropane,
C-αD-xylopyranoside-2-hydroxypropane,
1-(C-β-D-fucopyranoside)propan-2-one,
1-(C-αD-fucopyranoside)propan-2-one,
1-(C-β-L-fucopyranoside)propan-2-one,
1-(C-αL-fucopyranoside)propan-2-one,
1-(C-β-D-fucopyranoside)-2-hydroxypropane,
1-(C-αD-fucopyranoside)-2-hydroxypropane,
1-(C-β-L-fucopyranoside)-2-hydroxypropane,
1-(C-αL-fucopyranoside)-2-hydroxypropane,
1-(C-β-D-glucopyranosyl)-2-hydroxypropane,
1-(C-αD-glucopyranosyl)-2-hydroxypropane,
1-(C-β-D-galactopyranosyl)-2-hydroxypropane,
1-(C-αD-galactopyranosyl)-2-hydroxypropane,
1-(C-β-D-fucofuranosyl)propan-2-one,
1-(C-αD-fucofuranosyl)propan-2-one,
1-(C-β-L-fucofuranosyl)propan-2-one,
1-(C-αL-fucofuranosyl)propan-2-one,
C-β-D-maltopyranoside-n-propane-2-one,
C-αD-maltopyranoside-n-propan-2-one,
C-β-D-maltopyranoside-2-hydroxypropane,
C-αD-maltopyranoside-2-hydroxypropane, their isomers and their mixtures.

According to one embodiment, C-β-D-xylopyranoside-2-hydroxypropane or C-αD-xylopyranoside-2-hydroxypropane and better still C-β-D-xylopyranoside-2-hydroxypropane can advantageously be used for the preparation of a composition according to the invention.

According to a specific embodiment, the C-glycoside compound can be C-β-D-xylopyranoside-2-hydroxypropane (or hydroxypropyl tetrahydropyrantriol) provided in the form of a solution containing 35% by weight of active material in propylene glycol.

Advantageously, the cosmetic active compound selected from C-glycosides of formula (1) is present in the composition in an amount ranging from 0.1 wt. % to 20 wt. %, preferably from 0.1 wt. % to 10 wt. %, preferably from 0.1 wt. % to 5 wt. %, relative to the total weight of the composition.

### Fatty Acid Ester of Polyglycerol

According to the first aspect, the composition of the present invention comprises at least one surfactant selected from fatty acid esters of polyglycerol.

Preferably, the fatty acid ester of polyglycerol (also knowns as polyglycerol fatty acid ester, PGFE) suitable for the present invention is the compound of formula (II),

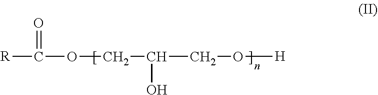

wherein:
R represents a linear or branched C₅-C₃₀ alkyl or alkenyl;
n is an integer ranging from 5 to 30.

Preferably, for the purpose of the present invention, in the formula (II),
R represents a linear or branched C₇-C₁₇ alkyl or alkenyl,
n is an integer ranging from 5 to 20, preferably from 5 to 16.

Examples of suitable PGFE surfactants are Polyglyceryl-10 Caprate, Polyglyceryl-10 Laurate, Polyglyceryl-10 Dilaurate, Polyglyceryl-10 Myristate, Polyglyceryl-10 Dimyristate, Polyglyceryl-10 Oleate, Polyglyceryl-10 Dioleate, Polyglyceryl-10 stearate, Polyglyceryl-10 Isostearate, Polyglyceryl-10 Diisostearate, Polyglyceryl-5 Laurate, Polyglyceryl-5 Dilaurate, Polyglyceryl-5 Myristate, Polyglyceryl-5 Trimyristate, Polyglyceryl-5 Oleate, or Polyglyceryl-5 Dioleate, Polyglyceryl-5 Stearate.

More particularly, the fatty acid esters of polyglycerol suitable for the present invention are compounds selected from the group consisting of Polyglyceryl-5 μlaurate, Polyglyceryl-5 Myristate, Polyglyceryl-5 Oleate, Polyglyceryl-5 Stearate, Polyglyceryl-10 Caprate, Polyglyceryl-10 Laurate, Polyglyceryl-10 Myristate, Polyglyceryl-10 Oleate, Polyglyceryl-10 stearate, Polyglyceryl-10 Isostearate, or a mixture thereof.

These compounds are commercially available. For example, mentions can be made to the compounds sold by the company Nikkol under the trade names: Sunsoft A-12E or Sunsoft A-121E-C(Polyglyceryl-5 μlaurate), Sunsoft A-14E (Polyglyceryl-5 Myristate), Sunsoft A-17E (Polyglyceryl-5 Oleate), Sunsoft A-18E (Polyglyceryl-5 Stearate), Sunsoft Q-10Y (Polyglyceryl-10 Caprate), Sunsoft Q-12Y (Polyglyceryl-10 Laurate), Sunsoft Q-14S (Polyglyceryl-10 Myristate), Nikkol Decaglyn 1-OV (Polyglyceryl-10 Oleate), and Sunsoft Q-18Y (Polyglyceryl-10 stearate).

In a particular embodiment, polyglyceryl-5 μlaurate is used in the composition of the present invention.

Advantageously, the surfactant selected from fatty acid esters of polyglycerol is present in the composition in an amount ranging from 0.1 wt. % to 10 wt. %, preferably from 0.2 wt. % to 8 wt. %, more preferably from 0.3 wt. % to 4 wt. %, relative to the total weight of the composition.

### Oxyalkylenated Fatty Acid Esters of Glycerol

According to the first aspect, the composition of the present invention comprises at least one surfactant selected from oxyalkylenated fatty acid esters of glycerol.

Particularly, the oxyalkylenated fatty acid ester of glycerol may be selected from saturated or unsaturated C₈-C₂₄ (especially C₈-C₁₈) fatty acid esters of oxyethylenated glyceryl ethers (which may comprise from 1 to 150 oxyethylene groups, in particularly, from 1 to 50 oxyethylene groups), for instance PEG-200 glyceryl monostearate sold under the name “Simulsol 200 ™” by the company SEPPIC; glyceryl stearate polyethoxylated with 30 ethylene oxide groups, for instance the product “Tagat S” sold under the company Goldschmidt; glyceryl oleate polyethoxylated with 30 ethylene oxide groups, for instance the product “Tagat O” sold by the company Goldschmidt; glyceryl cocoate polyethoxylated with 30 ethylene oxide groups, for instance the product “Varionic LI 13” sold by the company Sherex; glyceryl isostearate polyethoxylated with 30 ethylene oxide groups, for instance the product “Tagat L” sold by the company Goldschmidt; and glyceryl laurate polyethoxylated with 30 ethylene oxide groups, for instance the product “Tagat I” form the company Goldschmidt.

Preferably, the oxyalkylenated fatty acid ester of glycerol is selected from saturated or unsaturated C₈-C₁₈ fatty acid esters of oxyethylenated glyceryl ethers comprising from 1 to 50 oxyethylene groups.

More particularly, the oxyalkylenated fatty acid esters of glycerol may be selected, for example, from oxyethylenated glyceryl cocoates comprising from 1 to 30 oxyethylene groups, such as PEG-7 glyceryl cocoate, sold under the name Tegosoft GC® by the company Goldschmidt or CETIOL HE by the company BASF, and PEG-30 glyceryl cocoate, sold under the name Rewoderm LI-63 by the company Goldschmidt; or a mixture thereof.

Advantageously, the surfactant selected from oxyalkylenated fatty acid esters of glycerol is present in the composition in an amount ranging from 0.05 wt. % to 5 wt. %, preferably from 0.1 wt. % to 4 wt. %, more preferably from 0.2 wt. % to 2 wt. %, relative to the total weight of the composition.

### Peeling Agent

Preferably, the composition of the present invention comprises a peeling agent selected from N-substituted aminosulfonic acid compounds and α-hydroxy acids.

Preferably, the N-substituted aminosulfonic acid compound is an amino derivative of an alkylsulfonic acid. Preferably, the alkyl group has 1 to 10 carbon atoms, more preferably 2 to 6 carbon atoms, and even more preferably from 2 to 3 carbon atoms. Amino derivatives of ethanesulfonic acid and propanesulfonic acid are particularly preferred.

Suitable N-substituted aminosulfonic acid compounds include, but are not limited to, N,N-bis[2-hydroxyethyl]-2-aminoethanesulfonic acid, N-2-hydroxyethylpiperazine-N′-2-ethanesulfonic acid, 3-[N-morpholino]propanesulfonic acid, piperazine-N,N′-bis[2-ethanesulfonic]acid, 3-[N-tris(hydroxymethyl)methylamino]-2-hydroxypropanesulfonic acid (pKa=7.6), 2-[N-morpholino]ethanesulfonic acid, N-(2-acetamido)-2-aminoethanesulfonic acid, N-tris(hydroxymethyl)methyl-2-aminoethanesulfonic acid, and hydroxyethylpiperazine ethane sulfonic acid. A mixture of these acids may also be used.

Hydroxyethylpiperazine ethane sulfonic acid is a particularly preferred N-substituted aminosulfonic acid compound.

If presents, advantageously, the N-substituted aminosulfonic acid compound is present in the composition in an amount ranging from 1 wt. % to 10 wt. %, preferably from 2 wt. % to 6 wt. %, relative to the total weight of the composition.

Preferably, α-hydroxy acids are monocarboxylic acids, dicarboxylic acids, tricarboxylic acids.

Preferred examples of α-hydroxy acids include glycolic acid, citric acid, lactic acid, tartaric acid, malic acid, and mandelic acid.

If presents, advantageously, the α-hydroxy acid is present in the composition in an amount ranging from 0.3 wt. % to 10 wt. %, preferably from 1 wt. % to 6 wt. %, relative to the total weight of the composition.

### Aqueous Phase

The cosmetic composition of the present invention comprises an aqueous phase.

Said aqueous phase comprises water.

Advantageously, water is present in the composition of the present invention in an amount ranging from 50% to 90% by weight, preferably from 60% to 85% by weight, relative to the total weight of the composition.

Preferably, the aqueous phase comprises an organic solvent miscible with water (at room temperature 25° C.) selected from glycols and polyols having from 2 to 20 carbon atoms, preferably from 2 to 10 carbon atoms, and preferentially having from 2 to 6 carbon atoms, such as glycerin, propylene glycol, butylene glycol, pentylene glycol, hexylene glycol, caprylyl glycol, dipropylene glycol, diethylene glycol; and mixtures thereof, so as to provide a hydration effect.

If presents, advantageously, the organic solvent miscible with water selected from glycols and polyols is present in the composition in an amount ranging from 0.5 wt. % to 20 wt. %, preferably from 1 wt. % to 10 wt. %, relative to the total weight of the composition.

Preferably, the continuous aqueous phase of the composition of the present invention comprises water and glycerin.

### Additional Cosmetic Active Ingredients

The composition of the present invention may comprise an additional cosmetic active ingredient in addition to the cosmetic active compound of formula (I) as defined previously.

As examples of cosmetic active ingredient, mention can be made of moisturizing agents such as protein hydrolysates; botanical extracts (such as Carica papaya fruit extract, hydrolyzed Opuntia ficus-indica flower extract); vitamins such as vitamin A (retinol), vitamin E (tocopherol), vitamin C (ascorbic acid), vitamin B5 (panthenol),vitamin B3 (niacinamide), and derivatives of said vitamins (in particular esters) and mixtures thereof; urea; caffeine; tightening agents; agents acting on the microcirculation, and mixtures thereof.

It is easy for the skilled in the art to adjust the amount of the additional cosmetic active ingredient based on the final use of the composition according to the present invention.

### Additional Adjuvants or Additives

The composition of the present invention may comprise may also contain conventional cosmetic adjuvants or additives, for instance fragrances, chelating agents (for example, phytic acid), preserving agents (for example, salicylic acid) and bactericides, thickeners (such as xanthan gum), pH regulators (for example, triethanolamine, citric acid and sodium hydroxide), and mixtures thereof.

The skilled in the art can select the amount of the additional adjuvants or additive so as not to adversely impact the final use of the composition according to the present invention.

According to a particularly preferred embodiment, the present invention provides a composition for caring for keratin materials comprising in an aqueous phase, relative to the total weight of the composition:
(i) from 0.1 wt. % to 5 wt. % of at least one cosmetic active compound selected from C-β-D-xylopyranoside-2-hydroxypropane, C-αD-xylopyranoside-2-hydroxypropane, and a mixture thereof;
(ii) from 0.3 wt. % to 4 wt. % of at least one surfactant selected from Polyglyceryl-5 laurate, Polyglyceryl-5 Myristate, Polyglyceryl-5 Oleate, Polyglyceryl-5 Stearate, Polyglyceryl-10 Caprate, Polyglyceryl-10 Laurate, Polyglyceryl-10 Myristate, Polyglyceryl-10 Oleate, Polyglyceryl-10 stearate, Polyglyceryl-10 Isostearate, and a mixture thereof;
(iii) from 0.2 wt. % to 2 wt. % of at least one surfactant selected from oxyethylenated glyceryl cocoates comprising from 1 to 30 oxyethylene groups; and
(iv) from 2 wt. % to 6 wt. % of N-substituted aminosulfonic acid compound.

### Galenic Form and Method

The composition of the present invention is in the form of cream, lotion, or hydrogel.

The composition of the present invention can be used for caring for keratin materials.

According to the second aspect, the present invention provides a non-therapeutic method for caring for keratin materials, comprising applying the composition according to the first aspect of the present invention to the keratin materials.

In some embodiments, the present invention provides a non-therapeutic method for antiaging of keratin materials, comprising applying the composition according to the first aspect of the present invention to the keratin materials.

In particular, the keratin material is the skin.

The following examples serve to illustrate the present invention without, however, being limiting in nature.

### EXAMPLES

Main raw materials used, trade names and supplier thereof are listed in Table 1.

TABLE 1

| INCI Name | Trade Name | Supplier |
|---|---|---|
| PEG-7GLYCERYL COCOATE | CETIOL HE | BASF |
| POLYGLYCERYL-5 LAURATE | SUNSOFT A-121E-C | TAIYO KAGAKU |
| HYDROXYPROPYL | MEXORYL SCN | CHIMEX (NOVEAL) |
| TETRAHYDROPYRANTRIOL | | |
| GLYCERIN | GLICENAT ® GC K MB | OXITENO |
| LACTIC ACID | L(+)-LACTIC ACID 90% | JUNGBUNZLAUER |
| | HEAT STABLE PERSONAL | |
| | CARE GRADE | |
| XANTHAN GUM | KELTROL ® CG-T | CP KELCO |
| SALICYLIC ACID | SALICYLIC ACID USP | CELLMARK |
| HYDROXYETHYLPIPERAZINE | HEPES-LUV | TAIWAN HOPAX |
| ETHANE SULFONIC ACID | | |
| HYDROLYZED OPUNTIA FICUS- | EXFOLACTIVE EL | SILAB |
| INDICA FLOWER EXTRACT (and) | | |
| ETHYLHEXYLGLYCERIN | | |
| CARICA PAPAYA FRUIT EXTRACT | GLYCEROLAT ® PAPAYA C | CEP (SOLABIA) |
| PHYTIC ACID | PHYTIC ACID 50% | TSUNO RICE FINE |
| | SOLUTION | CHEMICALS |

### Invention Example 1 and Comparative Examples 1-4

Compositions of invention example (IE) 1 and comparative examples (CE) 1-4 were prepared based on the amounts given in Table 2. The amounts are given in % by weight of active ingredient relative to the total weight of the composition.

TABLE 2

| Components | IE1 | CE1 | CE2 | CE3 | CE4 |
|---|---|---|---|---|---|
| PEG-7 GLYCERYL COCOATE | 0.5 | 1.5 | — | — | — |
| POLYGLYCERYL-5 LAURATE | 1 | — | — | — | 1.5 |
| LACTIC ACID | — | — | 3 | — | — |
| HYDROXYPROPYL | 3.15 | 3.15 | 3.15 | 3.15 | 3.15 |
| TETRAHYDROPYRANTRIOL | | | | | |
| XANTHAN GUM | 0.4 | 0.4 | 0.4 | 0.4 | 0.4 |
| SALICYLIC ACID | 0.2 | 0.2 | 0.2 | 0.2 | 0.2 |
| GLYCERIN | 3 | 3 | 3 | 3 | 3 |
| HYDROLYZED OPUNTIA FICUS- | 4 | 4 | 4 | 4 | 4 |
| INDICA FLOWER EXTRACT (and) | | | | | |
| ETHYLHEXYLGLYCERIN | | | | | |
| CARICA PAPAYA FRUIT EXTRACT | 1 | 1 | 1 | 1 | 1 |
| PHYTIC ACID | 0.3 | 0.3 | 0.3 | 0.3 | 0.3 |
| WATER | QS100 | QS100 | QS 100 | QS 100 | QS 100 |

Composition of invention example 1 represents compositions according to the present invention.

Composition of comparative example 1 does not comprise fatty acid ester of polyglycerol.

Composition of comparative example 2 comprises lactic acid rather than fatty acid ester of polyglycerol and oxyalkylenated fatty acid ester of glycerol.

Composition of comparative example 3 does not comprise fatty acid ester of polyglycerol and oxyalkylenated fatty acid ester of glycerol.

Composition of comparative example 4 does not comprise oxyalkylenated fatty acid ester of glycerol.

### Preparation Process:

The compositions listed above were prepared as follows, taking the composition of invention formula 1 as an example:
1). Dissolve glycerin, and salicylic acid in water at 70° C. with stirring until them were totally dissolved;
2). Add xanthan gum with stirring until no lump observed, then cool down to room temperature; and
3). Add the remaining materials one by one with stirring.

### Evaluation

The penetration of hydroxypropyl tetrahydropyrantriol for each of compositions of invention example 1, comparative examples 2 and 3 was evaluated by the protocol from the company Biogalenys. In particular, the protocol is as follow:

A whole frozen human skin obtained from abdominal plastic surgery was used in this study. Each skin sample had an application area of 2 cm² after assembly on static Franz-type diffusion cell. 5 mg/cm² of each composition of invention examples and comparative examples were applied onto the skin surface for 16 hours. Three different donors with 3 samples per donor were used. Thus, 9 results were generated on one formula and then averaged in this test.

After 16 hours of exposure time, skin surface was washed with a solution containing 5% of sodium lauryl ether sulfate (used as cleanser), then rinsed with water, and at last dried with cotton swab.

The cutaneous distribution profile of hydroxypropyl tetrahydropyrantriol was determined by LC/MSMS analysis in the different compartments (skin excess, stratum corneum, epidermis, dermis and receptor fluid). LC/MSMS analysis was selected for its great sensibility and specificity.

The results of the bioavability of hydroxypropyl tetrahydropyrantriol of the compositions according to invention examples and comparative examples were listed in Table 3.

TABLE 3

| Compartment | IE. 1 (n = 9) | CE. 2(n = 9) | CE. 3(n = 9) |
|---|---|---|---|
| | μg/cm²(mean) | | |
| Skin excess | 133.62 | 140.03 | 142.3 |
| Stratum corneum(SC) | 2 | 1.62 | 2.26 |
| Skin (epidermis + dermis) | 2.21 | 0.99 | 1.05 |
| Receptor fluid (RF) | 0.67 | 0.28 | 0.46 |
| epidermis + dermis + RF | 2.87 | 1.27 | 1.51 |
| | % of the applied dose (mean) | | |
| Skin excess | 85.17 | 89.9 | 92.97 |
| Stratum corneum(SC) | 1.27 | 1.04 | 1.47 |
| Skin (epidermis + dermis) | 1.4 | 0.63 | 0.69 |
| Receptor fluid (RF) | 0.53 | 0.18 | 0.3 |
| epidermis + dermis + RF | 1.93 | 0.81 | 0.99 |
| Total Recovery | 88.37 | 91.75 | 95.43 |

It can be seen from Table 3 that the combination of a fatty acid ester of polyglycerol and an oxyalkylenated fatty acid ester of glycerol can boost the penetration of hydroxypropyl tetrahydropyrantriol to epidermis and dermis effectively.

In addition, the penetration of cosmetic active compound in each composition prepared in Example 1, comparative examples 1 and 4 was characterized as follows.

### i) Preparation of Skin Tissue Samples

6 ul of composition of each formula was applied evenly on 0.8 cm×0.8 cm porcine skin (pig ear skin from food industry), corresponding to 9 mg/cm². The porcine skin sample was then emerged on insert membrane with PBS underneath, followed by 37° C. incubation at 95% RH for 2 hours. Treated sample was embedded in an OCT Tissue Freezing Medium, then frozen and cryo-sectioned into 20 μm thickness. It was further placed on a CaF2 substrate for Raman confocal scanning. Three porcine samples were prepared for each formula. A Raman confocal mapping was acquired of each treated sample.

### ii) Raman Spectroscopy

A LabRam HR Evolution (Horiba Jobin-Yvon, Villeneuve-d' Ascq, France) Raman confocal microscope was used. Raman spectrum was obtained using a 532 nm DPSS laser with a power of 8 mW on the sample, coupled with a ×50 LM Plan objective (Olympus, NA 0.75, Rungis, France). The confocal hole was set at 100 μm diameter for all measurements. The system was spectrally calibrated to the 520.7 cm⁻¹ spectral line of silicon before the test. Detection was facilitated by dispersing Raman-shifted radiation onto a charge-coupled device (CCD) detector using a grating of 600 μlines/mm.

For pure cosmetic active ingredients, single point spectra were acquired with 25% laser intensity and 10 seconds acquisition time, for 400-2000 cm⁻¹ spectral range.

For mapping in the sample evaluation, the step size was 3 μm in both X and Y direction. The acquisition areas were 18×150 μm. For each spot, 50% laser intensity and 5 seconds acquisition time per spectrum was used. Spectral range was 400-2000 cm⁻¹.

iii) Data Analysis

Non-negative constrained least square (NCLS) analysis was performed using Matlab. Before statistical analysis, Raman spectra were subjected to a linear baseline correction. Cosmetic active ingredient spectral of 400-2000 cm⁻¹ fingerprints were used for analysis.

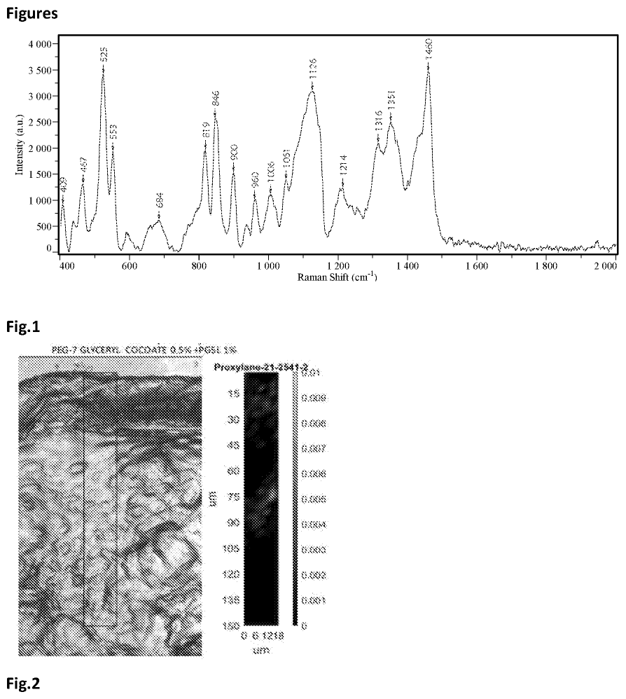

FIG. 1 shows the Raman spectra of 400-2000 cm⁻¹ for hydroxypropyl tetrahydropyrantriol.

A simplified description of the NCLS outcome can be defined as:

$$Ss = ( SR 1 * C 1 ) + ( SR 2 + C 2 ) + … + ( SRi * Ci ) + R * CR$$

Ss: Acquired Raman signal of one pixel on treated porcine skin sample;
SRi: Raman signal of each suppositional ingredient (PCA component) in untreated porcine;
R: Raman signal of pure cosmetic active ingredient;
Ci: coefficient of each suppositional ingredient (PCA component) in the exact pixel;
CR: coefficient of cosmetic active ingredient in the exact pixel; The computational co-efficient index of active ingredients can be used to generate the distribution profile of ACIs from outer skin stratum corneum to the deeper dermis part.

FIG. 2 shows the penetration profile of hydroxypropyl tetrahydropyrantriol for the composition of invention formula 1.

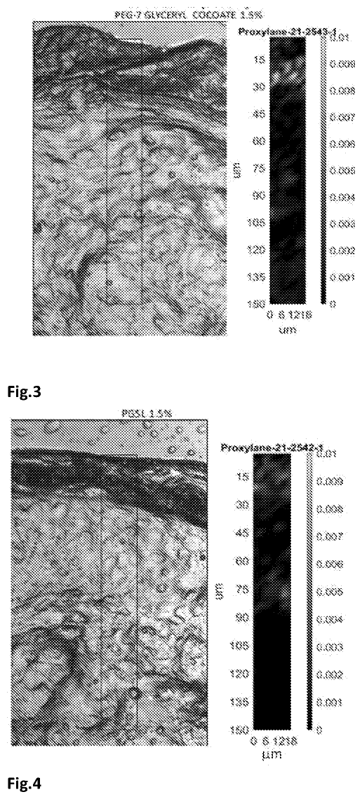

FIG. 3 shows the penetration profile of hydroxypropyl tetrahydropyrantriol for the composition of comparative formula 1; and

FIG. 4 shows the penetration profile of hydroxypropyl tetrahydropyrantriol for the composition of comparative formula 4.

It can be seen from FIGS. 2-4 that the penetration of hydroxypropyl tetrahydropyrantriol into the skin after topical application for composition of invention example 1 and composition of comparative example 4 are similar, and better than that for composition of comparative example 1.

The stickiness of composition of invention example 1 and composition of comparative example 4 was evaluated as follows.

5 skincare formulation scientists applied 50 μl of the composition tested on an area of 6.3 cm×6.3 cm of arm skin with two washed fingers. Then the stickiness was scored during application and after application according to the following standards and averaged:
1=not sticky,
2=acceptable stickiness for skincare products,
3=very sticky.

The results of the stickiness of the compositions of invention example 1 and comparative example 4 were listed in Table 4.

TABLE 4

| | Inv. 1 | Comp. 4 |
|---|---|---|
| Stickiness | 2 | 3 |

In summary, composition according to the present invention can deliver the active ingredient hydroxypropyl tetrahydropyrantriol effectively and present a good skin sensory.

1: A composition for caring for keratin materials comprising in an aqueous phase:
(i) at least one cosmetic active compound selected from C-glycosides of formula (I): 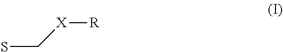
in which:
R represents a saturated C₁ to C₁₀, alkyl radical which can optionally be substituted by at least one radical selected from OH, COOH or COOR″₂, with R″₂ being a saturated C₁-C₄ alkyl radical,
S represents a monosaccharide or a polysaccharide comprising up to 20 sugar units, in pyranose and/or furanose form and of the L and/or D series, it being possible for the said monosaccharide or polysaccharide to be substituted by a hydroxyl group which is necessarily free and optionally one or more optionally protected amine functional group(s), and
X represents a radical selected from the group consisting of —CO—, —CH(OH)—, —CH(NH₂)—, —CH(NHCH₂CH₂CH₂OH)—, —CH(NHPh)- and —CH(CH₃)— groups,
the S—CH₂—X bond represents a bond of C-anomeric nature, which can be α or β,
and also their physiologically acceptable salts, their solvates, such as the hydrates, and their optical and geometrical isomers;
(ii) at least one surfactant selected from fatty acid esters of polyglycerol; and
(iii) at least one surfactant selected from oxyalkylenated fatty acid esters of glycerol.

2: The composition according to claim 1,
wherein the cosmetic active compound is selected from the group consisting of
C-β-D-xylopyranoside-n-propane-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-αD-fucopyranoside)propan-2-one,
1-(C-β-L-fucopyranoside)propan-2-one,
1-(C-αL-fucopyranoside)propan-2-one,
1-(C-β-D-fucopyranoside)-2-hydroxypropane,
1-(C-αD-fucopyranoside)-2-hydroxypropane,
1-(C-β-L-fucopyranoside)-2-hydroxypropane,
1-(C-αL-fucopyranoside)-2-hydroxypropane,
1-(C-β-D-glucopyranosyl)-2-hydroxypropane,
1-(C-αD-glucopyranosyl)-2-hydroxypropane,
1-(C-β-D-galactopyranosyl)-2-hydroxypropane,
1-(C-αD-galactopyranosyl)-2-hydroxypropane,
1-(C-β-D-fucofuranosyl)propan-2-one,
1-(C-αD-fucofuranosyl)propan-2-one,
1-(C-β-L-fucofuranosyl)propan-2-one,
1-(C-αL-fucofuranosyl)propan-2-one,
C-β-D-maltopyranoside-n-propane-2-one,
C-αD-maltopyranoside-n-propan-2-one,
C-β-D-maltopyranoside-2-hydroxypropane,
C-αD-maltopyranoside-2-hydroxypropane,
their isomers and their mixtures.

3: The composition according to claim 1, wherein the cosmetic active compound is present in an amount ranging from 0.1 wt. % to 20 wt. % relative to the total weight of the composition.

4: The composition according to claim 1, wherein the fatty acid ester of polyglycerol is the compound of formula (II), 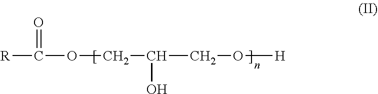
wherein:
R represents a linear or branched C₅-C₃₀ alkyl or alkenyl; and
n is an integer ranging from 5 to 30 .

5: The composition according to claim 4, wherein the fatty acid ester of polyglycerol is selected from the group consisting of Polyglyceryl-5 μlaurate, Polyglyceryl-5 Myristate, Polyglyceryl-5 Oleate, Polyglyceryl-5 Stearate, Polyglyceryl-10 Caprate, Polyglyceryl-10 Laurate, Polyglyceryl-10 Myristate, Polyglyceryl-10 Oleate, Polyglyceryl-10 stearate, Polyglyceryl-10 Isostearate, er and a mixture thereof.

6: The composition according to claim 1, wherein the surfactant selected from fatty acid esters of polyglycerol is present in an amount ranging from 0.1 wt. % to 10 wt. % relative to the total weight of the composition.

7: The composition according to claim 1, wherein the oxyalkylenated fatty acid ester of glycerol is selected from the group consisting of saturated or unsaturated C₈-C₂₄ fatty acid esters of oxyethylenated glyceryl ethers comprising from 1 to 150 oxyethylene groups.

8: The composition according to claim 1, wherein the surfactant selected from oxyalkylenated fatty acid esters of glycerol is present in an amount ranging from 0.05 wt. % to 5 wt. % relative to the total weight of the composition.

9: The composition according to claim 1, wherein the composition comprises a peeling agent selected from the group consisting of N-substituted aminosulfonic acid compounds and α-hydroxy acids.

10: The composition according to claim 9, wherein the composition comprises a N-substituted aminosulfonic acid compound selected from the group consisting of N,N-bis[2-hydroxyethyl]-2-aminoethanesulfonic acid, N-2-hydroxyethylpiperazine-N′-2-ethanesulfonic acid, 3-[N-morpholino]propanesulfonic acid, piperazine-N,N′-bis[2-ethanesulfonic]acid, 3-[N-tris(hydroxymethyl)methylamino]-2-hydroxypropanesulfonic acid (pKa=7.6), 2-[N-morpholino]ethanesulfonic acid, N-(2-acetamido)-2-aminoethanesulfonic acid, N-tris(hydroxymethyl)methyl-2-aminoethanesulfonic acid, and hydroxyethylpiperazine ethane sulfonic acid.

11: The composition according to claim 10, wherein the N-substituted aminosulfonic acid compound is present in an amount ranging from 1 wt. % to 10 wt. % relative to the total weight of the composition.

12: The composition according to claim 9, wherein the composition comprises an α-hydroxy acid selected from the group consisting of glycolic acid, citric acid, lactic acid, tartaric acid, malic acid, and mandelic acid.

13: The composition according to claim 1, wherein the α-hydroxy acid is present in an amount ranging from 0.3 wt. % to 10 wt. % relative to the total weight of the composition.

14: The composition according to claim 1, comprising in an aqueous phase, relative to the total weight of the composition:
(i) from 0.1 wt. % to 5 wt. % of at least one cosmetic active compound selected from the group consisting of C-β-D-xylopyranoside-2-hydroxypropane, C-α-D-xylopyranoside-2-hydroxypropane, and a mixture thereof;
(ii) from 0.3 wt. % to 4 wt. % of at least one surfactant selected from the group consisting of Polyglyceryl-5 μlaurate, Polyglyceryl-5 Myristate, Polyglyceryl-5 Oleate, Polyglyceryl-5 Stearate, Polyglyceryl-10 Caprate, Polyglyceryl-10 Laurate, Polyglyceryl-10 Myristate, Polyglyceryl-10 Oleate, Polyglyceryl-10 stearate, Polyglyceryl-10 Isostearate, and a mixture thereof;
(iii) from 0.2 wt. % to 2 wt. % of at least one surfactant selected from the group consisting of oxyethylenated glyceryl cocoates comprising from 1 to 30 oxyethylene groups; and
(iv) from 2 wt. % to 6 wt. % of N-substituted aminosulfonic acid compound.

15: A non-therapeutic method for caring for keratin materials, comprising applying the composition according to claim 1 to the keratin materials.
